(19) United States
(12) Patent Application Publication
(10) Pub. No.: US 2008/0287511 A1
(43) Pub. Date: Nov. 20, 2008

(54) Anthelmintic Imidazol-Thiazole Derivates

(76) Inventors: Jan Heeres, Vosselaar (BE); Paulus Joannes Lewi, Turnhout (BE); Paul Adriaan Jan Janssen, Vosselaar (BE); Kathleen Marie Jeanne Alice Vlaminck, Brugge-Sint Kruis (BE); Pierre Josef Hektor Valere Ottevaere, Beerse (BE); Oscar Franz Joseph Vanparijs, Vosselaar (BE); Frank Xavier Jozef Herwig Arts, Brasschaat (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/814,617

(22) PCT Filed: Jan. 26, 2006
(86) PCT No.: PCT/EP06/50460

§ 371 Date: Jul. 24, 2007

(30) Foreign Application Priority Data
Jan. 28, 2005 (EP) 05100580.9

(51) Int. Cl. A61K 31/429; C07D 513/02; A61P 33/14

(52) U.S. Cl. 514/368; 548/155

(57) ABSTRACT

The present invention relates to the novel anthelmintic compound tetrahydro-furan-2-carboxylic acid-[3-(2,3,5,6-tetrahydro-imidazo[2,1-b]thiazol-6-yl)-phenyl]-amide and the pharmaceutically acceptable acid addition salts and the stereochemically isomeric forms thereof, as well as pharmaceutical compositions comprising said novel compound, processes for preparing said compound and compositions, and the use thereof as a medicine, in particular in treatment, control and prevention of endo- and ectoparasite infections in warm-blooded animals.

The present invention is concerned with novel anthelmintic tetramisole derivatives and the pharmaceutically acceptable acid addition salts thereof, compositions comprising said novel compounds, processes for preparing said compounds and compositions, and the use thereof as a medicine, in particular in treatment, control and prevention of endo- and ectoparasite infections in warm-blooded animals.

Tetramisole and levamisole are very well known anthelmintics having the following structure:

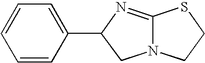

(tetramisole is the racemic dl-form, levamisole is the enantiomeric pure l-form).

One of the most popular anthelmintics is levamisole which has been widely used to control nematode parasites in farm animals, in particular in sheep and cattle. However the development of nematodes resistant to anthelmintics has become a major problem in farm animals, particularly with the nematode Haemonchus contortus. Moreover multi-resistant strains have been found in field circumstances that have developed resistance against widely used anthelmintics such as levamisole, mebendazole and ivermectin. Hence there is a need to find new anthelmintic agents having anthelmintic activity against levamisole resistant and multi-resistant nematodes.

Other tetramisole anthelmintics have been disclosed in, e.g. U.S. Pat. No. 4,014,892 which exemplifies furan-2-carboxylic acid [3-(2,3,5,6-tetrahydro-imidazo[2,1-b]thiazol-6-yl)-phenyl]-amide as compound (163) and (tetrahydrofuran-2-ylmethyl)-[3-(2,3,5,6-tetrahydro-imidazo[2,1-b]thiazol-6-yl)-phenyl]-amine as compound (110). The latter compound is also disclosed in GB-1,365,515 as compound (81).

The present invention relates to novel compounds of formula (I)

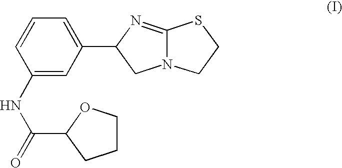

the pharmaceutically acceptable acid addition salts and the stereochemically isomeric forms thereof.

As demonstrated in pharmacological example C.1 the compounds of the present invention have an unexpected better anthelmintic activity against a multi-resistant strain of Haemonchus contortus (resistant against levamisole, mebendazole, ivermectin and closantel) than the art known compounds furan-2-carboxylic acid [3-(2,3,5,6-tetrahydro-imidazo[2,1-b]thiazol-6-yl)-phenyl]-amide (referenced to as compound (A) in the description) and (tetrahydrofuran-2-ylmethyl)-[3-(2,3,5,6-tetrahydro-imidazo[2,1-b]thiazol-6-yl)-phenyl]-amine (referenced to as compound (B) in the description).

The pharmaceutically acceptable acid addition salts as mentioned hereinabove are meant to comprise the therapeutically active non-toxic acid addition salt forms that the compounds of formula (I) are able to form. These pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

Conversely said salt forms can be converted by treatment with an appropriate base into the free base form.

The term “stereochemically isomeric forms” as used hereinbefore defines all the possible isomeric forms which the compounds of formula (I) may possess. Unless otherwise mentioned or indicated, the chemical designation of compounds of formula (I) denotes the mixture of all possible stereochemically isomeric forms, said mixtures containing all diastereomers and enantiomers of the basic molecular structure. More in particular, stereogenic centers may have the R- or S-configuration. The compounds of formula (I) have two chiral carbon atoms, as indicated by an asterisk in the structure below

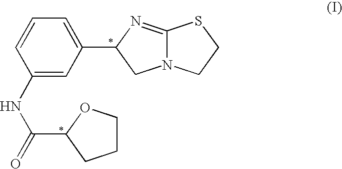

giving a total of 4 different stereoisomers. All 4 individual stereochemically isomeric forms of the compounds of formula (I), and every possible mixture thereof, are obviously intended to be embraced within the scope of this invention.

The absolute stereochemical configuration of the compounds of formula (I) and of the intermediates used in their preparation may easily be determined by those skilled in the art while using well-known methods such as, for example, X-ray diffraction.

Furthermore, some compounds of formula (I) and some of the intermediates used in their preparation may exhibit polymorphism. It is to be understood that the present invention encompasses any polymorphic forms possessing properties useful in the treatment of the conditions noted hereinabove.

A particular group of compounds are those compounds of formula (I-a) which are defined as compounds of formula (I) having the (S)-configuration at the 6-position of the 2,3,5,6-tetrahydro-imidazo[2,1-b]thiazole moiety.

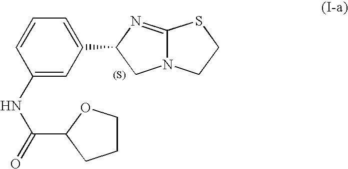

A preferred compound is (2R)-tetrahydro-furan-2-carboxylic acid (6S)-[3-(2,3,5,6-tetrahydro-imidazo[2,1-b]thiazol-6-yl)-phenyl]-amide.

Compounds of formula (I) can in general be prepared by reacting an intermediate of formula (V) dissolved in a dioxane solution acidified with HCl with an intermediate of formula (IV) in a suitable reaction-inert solvent such as acetonitrile.

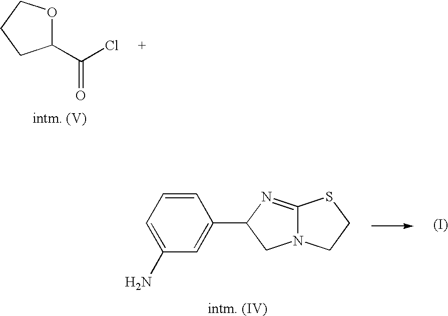

Intermediates of formula (IV) can be prepared as outlined below. The commercially available starting compound 2-bromo-1-(3-nitrophenyl)-ethanone acid is reacted with 4,5-dihydrothiazolamine and the obtained intermediate (I) is then reduced with an appropriate reducing agent such as sodium borohydride in a suitable solvent such as ethanol yielding intermediate (II). Intermediate (II) is then treated with thionyl chloride in a reaction-inert solvent such as dichloroethane whereby the 2,3,5,6-tetrahydro-imidazo[2,1-b]thiazolyl ring of intermediate (III) is formed. Intermediate (III) is first converted into its HCl addition salt before its nitro group is reduced to an amino group using art-known reduction agents such as iron powder and aqueous NH₄Cl or SnCl₂ thereby yielding intermediate (IV).

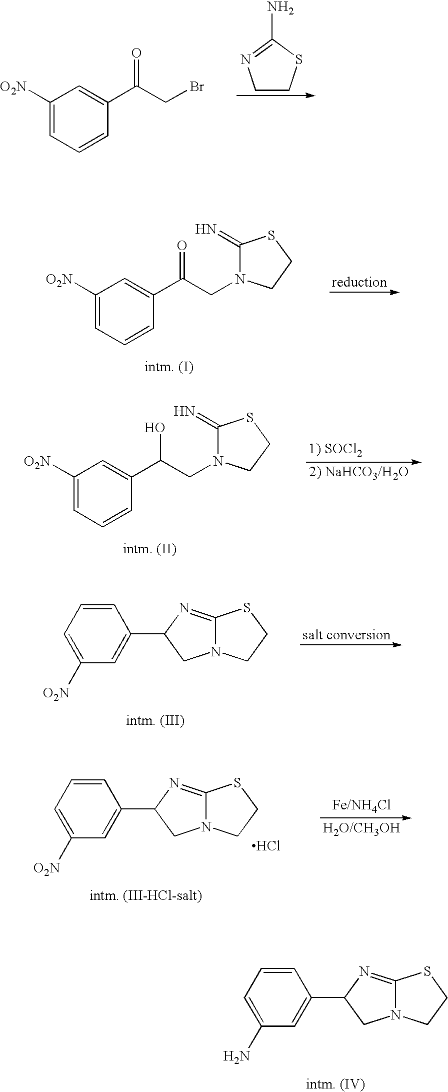

Intermediate (V) is prepared by converting tetrahydro-2-furancarboxylic acid into its acyl chloride analogue by reacting it with oxalylchloride. Intermediate (V) can also be prepared as its stereoisomeric pure (R)— or (S)-enantiomer by starting from either (R)- or (S)— tetrahydro-2-furancarboxylic acid respectively.

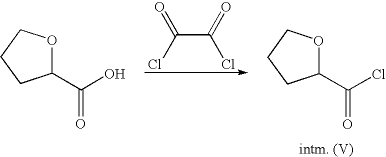

The intermediate of formula (III) can be separated into its (+)- or (−)-stereoisomers using art-known separation techniques such as liquid chromatography using a chiral stationary phase.

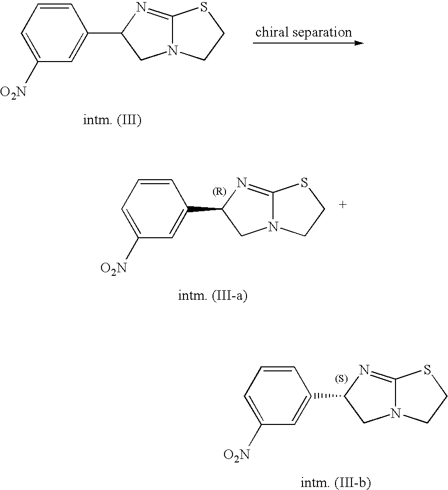

The compounds of formula (I) as prepared in the hereinabove described processes may be synthesized in the form of racemic mixtures of enantiomers which can be separated from one another following art-known resolution procedures. Those compounds of formula (I) that are obtained in racemic form may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

The compounds of formula (I), the pharmaceutically acceptable salts and stereo-isomeric forms thereof possess favorable anthelmintic activity. Therefore the present compounds of formula (I) are useful as a medicine in treatment, control and prevention of endo- and ectoparasite infections in warm-blooded animals.

Endo- and ectoparasites include Nemathelminthes such as Amidostomum, Ancylostoma, Angiostrongylus, Anisakis, Ascaris, Brugia, Bunostomum, Capillaria, Chabertia, Cooperia, Cyathostomum, Cylicocyclus, Dictyocaulus (lungworm), Dipetalonema, Dirofilaria (heartworm), Dracunculus, Elaeophora, Gaigeria, Globocephalus urosubulatu, Haemonchus, Heterakis, Hyostrongylus, Metastrongylus (lungworm), Muellerius (lungworm), Necator americanus, Nematodirus, Neoascaris, Oesophagostomum, Onchocerca, Ostertagia, Oxyuris, Parascaris, Protostrongylus (lungworm), Setaria, Stephanofilaria, Strongyloides, Strongylus, Syngamus, Teladorsagia, Toxascaris, Toxocara, Trichinella, Trichostrongylus, Trichuris, Uncinaria stenocephala, and Wuchereria bancrofti.

Warm-blooded animals as used throughout this text include both human and non-human animals such as farm animals (e.g. sheep, cattle, swine, goats or horses), domestic animals (e.g. dogs, cats, or cavias) as well as wild animals held in captivity and birds (e.g. poultry).

In view of the utility of the compounds of formula (I), it follows that the present invention also provides a method of treating, controlling and preventing endo- and ecto-parasite infections in warm-blooded animals. This method comprises administering to a warm-blooded animal in need thereof a therapeutically effective amount of a compound of formula (I).

The term “therapeutically effective amount of a compound of formula (I)” as used herein, means that amount of compound of formula (I) that elicits the biological or medicinal response in the warm-blooded animal that is being sought by the physician or veterinarian, which includes alleviation of the symptoms of the condition being treated. The therapeutically effective amount can be determined using routine optimization techniques and is dependent upon the particular condition to be treated, the condition of the warm-blooded animal, the route of administration, the formulation, and the judgment of the practitioner and other factors evident to those skilled in the art. A therapeutically effective amount may be achieved by multiple dosing.

Additionally the present invention provides pharmaceutical compositions comprising at least one pharmaceutically acceptable carrier and a therapeutically effective amount of a compound of formula (I).

For use in warm-blooded animals, including humans, the compounds of formula (I) can be administered alone, but will generally be administered in admixture with a pharmaceutically or veterinary acceptable diluent or carrier selected with regard to the intended route of administration and standard pharmaceutical practice. For example, they can be administered orally, including sublingually, in the form of tablets containing such excipients as starch or lactose, or in capsules or ovules either alone or in admixture with excipients, or in the form of elixirs, solutions or suspensions containing flavouring or colouring agents. The compounds of formula (I) could be incorporated into capsules, tablets or boluses for targeting the colon or duodenum via delayed dissolution of said capsules, tablets or boluses for a particular time following oral administration. The compounds of formula (I) can be injected parenterally, for example, intravenously, intramuscularly or subcutaneously. For parenteral administration, they are best used in the form of a sterile aqueous solution or suspension that may contain other substances, for example, enough salt or glucose to make the solution isotonic with blood. The compounds of formula (I) can be administered topically, in the form of sterile creams, gels, pour-on or spot-on formulations, suspensions, lotions, ointments, dusting powders, sprays, drug-incorporated dressings or via a skin patch. For example the compounds of formula (I) can be incorporated into a cream consisting of an aqueous or oily emulsion of polyethylene glycols or liquid paraffin, or they can be incorporated into an ointment consisting of a white wax soft paraffin base, or as hydrogel with cellulose or polyacrylate derivatives or other viscosity modifiers, or as a dry powder or liquid spray or aerosol with butane/propane, HFA or CFC propellants, or as a drug-incorporated dressing either as a tulle dressing, with white soft paraffin or polyethylene glycols impregnated gauze dressings or with hydrogel, hydrocolloid, alginate or film dressings. The compounds of formula (I) could also be administered intra-ocularly as an eye drop with appropriate buffers, viscosity modifiers (e.g. cellulose derivatives), preservatives (e.g. benzalkonium chloride (BZK)) and agents to adjust tenicity (e.g. sodium chloride). Such formulation techniques are well-known in the art. All such formulations may also contain appropriate stabilizers and preservatives.

For veterinary use, compounds can be administered as a suitably acceptable formulation in accordance with normal veterinary practice and the veterinarian will determine the dosing regimen and route of administration which will be most appropriate for a particular animal.

For topical application dip, spray, powder, dust, pour-on, spot-on, emulsifiable concentrate, jetting fluid, shampoos, collar, tag or harness may be used. Such formulations are prepared in a conventional manner in accordance with standard veterinary and pharmaceutical practice. Thus capsules, boluses or tablets may be prepared by mixing the active ingredient with a suitable finely divided diluent or carrier, additionally containing a disintegrating agent and/or binder such as starch, lactose, talc, or magnesium stearate. A drench formulation may be prepared by dispersing the active ingredients in an aqueous solution together with dispersing or wetting agents and injectable formulations may be prepared in the form of a sterile solution or emulsion. Pour-on or spot-on formulations may be prepared by dissolving the active ingredients in an acceptable liquid carrier vehicle, such as butyl digol, liquid paraffin or non-volatile ester with or without addition of a volatile component such as isopropanol.

Alternatively, pour-on, spot-on or spray formulations can be prepared by encapsulation to leave a residue of active agent on the surface of the animal. These formulations will vary with regard to the weight of active compound depending on the species of host animal to be treated, the severity and type of infection and type and body weight of the host. The formulations comprising a compound of formula (I) may be administered continuously, particularly for prophylaxis by known methods.

As an alternative the combinations may be administered with the animal feedstuff and for this purpose a concentrated feed additive or premix may be prepared for mixing with the normal animal feed.

For human use the compounds of formula (I) are administered as a pharmaceutically acceptable formulation in accordance with normal medical practice.

The compounds of formula (I) may be used in conjunction with other anthelmintic or antiparasitic agents so as to widen the spectrum of action or to prevent the buildup of resistance. Other anthelmintic agents are for example avermectines and milbemycines such as abamectin, cydectin, doramectin, eprinomectin, ivermectin, milbemycin, milbemycin D, milbemycin oxime, moxidectin, selamectin, and the like; benzimidazoles such as albendazole, cambendazole, fenbendazole, flubendazole, mebendazole, oxfendazole, parbendazole, oxibendazole and cyclobendazole; pro-benzimidazoles such as febantel, thiophanate and netobimin; salicylanilides such as closantel and niclosamide; imidazothiazoles such as butamisole and levamisole; tetrahydropyrimidines such as morantel, pyrantel en pyrantel pamoate; hexahydro-pyrazinoisoquinolines such as praziquantel; and macrolides produced by fermentation of Saccharopolyspora spinosa such as spinosyn A, spinosyn D or spinosad.

Those skilled in the treatment of helminthiasis will easily determine the therapeutically effective amount of a compound of formula (I) from the test results presented hereinafter. In general it is contemplated that a therapeutically effective dose will be from about 0.1 mg/kg to about 20 mg/kg of body weight, more preferably from about 1 mg/kg to about 10 mg/kg of body weight of the warm-blooded animal to be treated. It may be appropriate to administer the therapeutically effective dose in the form of two or more sub-doses at appropriate intervals throughout the day.

The exact dosage and frequency of administration depends on the particular compound of formula (I) used, the particular condition being treated, the severity of the condition being treated, the age, weight and general physical condition of the particular warm-blooded animal as well as the other medication (including the above-mentioned additional anthelmintic or antiparasitic agents), the warm-blooded may be taking, as is well known to those skilled in the art. Furthermore, said effective daily amount may be lowered or increased depending on the response of the treated animal and/or depending on the evaluation of the physician or veterinarian prescribing the compounds of the instant invention. The effective daily amount ranges mentioned hereinabove are therefore only guidelines.

### EXPERIMENTAL PART

### A. Synthesis of the Intermediates

### Example A.1

### Preparation of

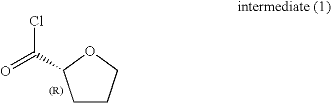

A solution of (+)-(R)-tetrahydro-2-furancarboxylic acid (6.65 ml) in dichloromethane (250 ml) was stirred under nitrogen at room temperature and then ethanedioyl dichloride (12.1 ml) and anhydrous dimethylformamide (3 drops) were added. The reaction mixture was stirred for 2.5 hours and then the solvent was evaporated and co-evaporated two times with toluene, yielding (2R)-tetrahydro-2-furancarbonyl chloride (intermediate 1).

In an analogous way racemic tetrahydro-2-furancarbonyl chloride was prepared starting from the commercially available tetrahydro-2-furancarboxylic acid and (2S)-tetrahydro-2-furancarbonyl chloride was prepared starting from commercially available (−)-(S)-tetrahydro-2-furancarboxylic acid.

### Example A.2

### a) Preparation of

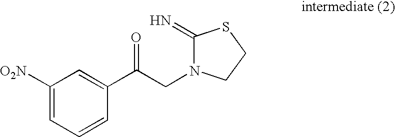

A suspension of 4,5-dihydrothiazolamine (1.95 mol) in 2-propanone (1000 ml) was stirred in a 4-necked flask with mechanical stirrer, thermometer and dripping funnel. Then a solution of 2-bromo-1-(3-nitrophenyl)ethanone (1.93 mol) in 2-propanone (2500 ml) was added dropwise while cooling on an ice bath to keep the temperature below 20° C. The reaction mixture was stirred overnight. The resulting precipitate was filtered off, washed with 2-propanone and dried, yielding 640 g of intermediate (2).

### b) Preparation of

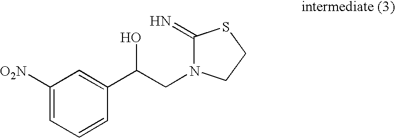

A suspension of intermediate (2) (1.85 mol) in ethanol (3000 ml) was chilled on an ice bath. Sodium borohydride (2.78 mol) was added portionwise and the reaction mixture was allowed to warm to room temperature overnight. A saturated NaHCO₃ solution (1000 ml) and water (500 ml) were added and after quenching dichloromethane was added. The resulting mixture was stirred for 1 hour and the aqueous layer was extracted with dichloromethane (4×1000 ml). The organic layers were combined, dried, filtered off and the solvent was evaporated, yielding 469 g of intermediate (3).

### c) Preparation of

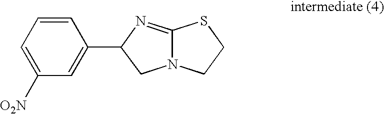

A suspension of intermediate (3) (0.34 mol) in 1,2-dichloroethane (3000 ml) was stirred at room temperature and a solution of thionyl chloride (0.68 mol) in 1,2-dichloroethane (150 ml) was added dropwise over 6 hours. The reaction mixture was stirred overnight and NaHCO₃ (1500 ml) was added carefully. The reaction mixture was warmed to 40° C., stirred for 6 hours and then the aqueous layer was removed. The organic layer was washed with a saturated NaHCO₃ solution (4×1000 ml), dried, filtered off and the solvent was evaporated. The obtained residue was dissolved in CH₂Cl₂/CH₃OH (95/5; 2000 ml) and stirred with silica gel (250 g). The silica gel was filtered off. The filtrate was filtered again over silica gel (1000 g) and the solvent was evaporated, yielding 253 g of intermediate (4).

### d) Preparation of

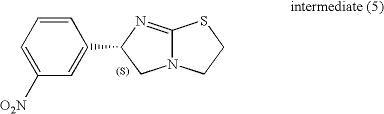

Intermediate (4) was separated into its enantiomers by chiral column chromatography over Chiralcel AS 1000 Å 20 μm (eluent: ethanol/heptane 30/70). The desired fractions comprising the (S)-enantiomer were collected and the solvent was evaporated, yielding (6S)-(3-nitro-phenyl)-2,3,5,6-tetrahydro-imidazo[2,1-b]thiazole (interm. 5). The fractions comprising the (R)-enantiomer were also collected and, after evaporation of the solvent, yielded (6R)-(3-nitro-phenyl)-2,3,5,6-tetrahydro-imidazo[2,1-b]thiazole (interm. 6).

### Example A.3

### Preparation of

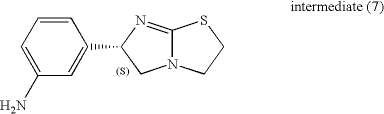

a) A solution of intermediate (5) (0.15 mol) in ethyl acetate was stirred at room temperature. A 1M solution of hydrochloric acid (0.199 mol) in diethylether (190 ml) was added dropwise. The resulting precipitate was filtered off and dried at low pressure, yielding 39.4 g of the hydrochloric acid salt of intermediate (5). b) Water (105 ml) and then methanol (35 ml) were added to a mixture of the hydrochloric acid salt of intermediate (5) (0.0122 mol), iron powder (0.061 mol) and ammonium chloride (0.061 mol). The reaction mixture was stirred and warmed to 70° C. for 30 minutes and was then allowed to cool to room temperature. 0.1N HCl (14 ml) was added and the resulting mixture was filtered over Kieselguhr. The Kieselguhr was washed with 0.01N HCl (175 ml) and with dichloromethane (175 ml). The obtained filtrate was stirred while a saturated NaHCO₃ solution (175 ml) and NaHCO₃ (10 g) were added. The organic layer was separated and the aqueous layer was extracted with dichloromethane (4×175 ml). The organic layers were combined, dried, filtered off and the solvent was evaporated, yielding 2.61 g of intermediate (7).

Using an analogous procedure but starting from intermediate (4), 3-(2,3,5,6-tetrahydro-imidazo[2,1-b]thiazol-6-yl)-phenylamine was prepared as intermediate (8).

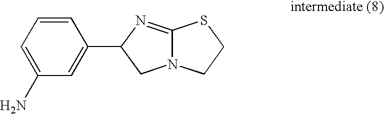

### Example A.4

### Preparation of

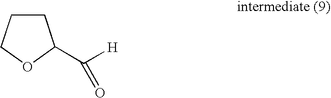

At −60° C., a solution of dimethylsulfoxide (36.5 ml, 0.514 mol) in dichloromethane (700 ml) was added dropwise to a 2M solution of oxalyl chloride (26.9 ml, 0.308 mol) in dichloromethane. The mixture was stirred for 30 minutes and then a solution of tetrahydrofurfuryl alcohol (25 ml, 0.257 mol) in dichloromethane (100 ml) was added dropwise. The mixture was stirred for 20 minutes and then triethylamine (181 ml, 1.29 mol) was slowly added. The reaction mixture was warmed up to room temperature and stirred for 30 minutes. The reaction mixture was filtered off and the residue was washed with dichloromethane (250 ml). The combined dichloromethane layers were washed with water (150 ml), dried and evaporated carefully at 25° C. To the residue was added diethyl ether, the precipitate was filtered off and washed with diethyl ether. The combined organic layers were evaporated carefully. The product was purified by bulb-to-bulb distillation (75° C., 20 mbar) yielding 12.6 gram of intermediate (9).

### B. Preparation of the Final Compounds

### Example B.1

### Preparation of

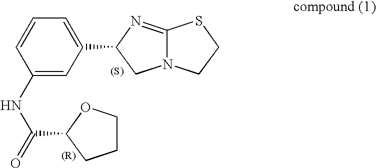

A 4M solution of hydrochloric acid in dioxane (16.43 ml) was added dropwise to a solution of intermediate (7) (0.066 mol) in anhydrous acetonitrile (500 ml). The resulting mixture was chilled on an ice-bath. A mixture of intermediate (1) (0.069 mol) in acetonitrile (100 ml) was added dropwise and the reaction mixture was allowed to reach room temperature overnight. Acetonitrile was evaporated. A saturated NaHCO₃ solution (500 ml) was added and the resulting mixture was extracted with dichloro-methane (3×500 ml). The organic layers were combined, dried, filtered off and the solvent was evaporated. The residue was purified by flash column chromatography (eluent: CH₂Cl₂/CH₃OH 95/5) over silica gel. The product fractions were collected and the solvent was evaporated, yielding 12.23 g of compound (1) (mp. 42-57° C.) (specific optical rotation OR=−3.77° (589 nm, c=0.4636 w/v %, methanol, 20° C.)).

Using the procedure as outlined above, the compounds (2), (3) and (4) were also prepared using respectively a combination of intermediate (8) and racemic tetrahydro-2-furancarbonyl chloride, or intermediate (7) and racemic tetrahydro-2-furancarbonyl chloride, or intermediate (7) and (2S)-tetrahydro-2-furancarbonyl chloride, as the starting materials.

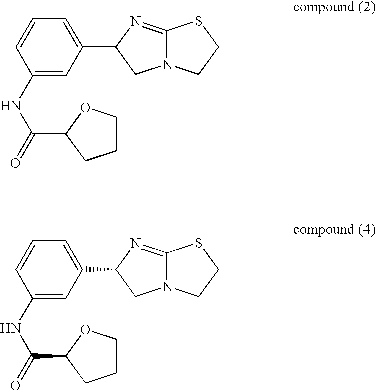

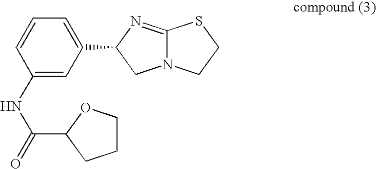

### Example B.2

### Preparation of

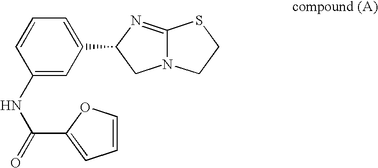

Intermediate (7) (470 mg) was dissolved in anhydrous acetonitrile (40 ml). A solution of 4 M HCl in dioxane (0.538 ml) was added dropwise. The mixture was cooled using an ice bath and a solution of 2-furancarbonyl chloride (0.213 ml) in acetonitrile (20 ml) was added dropwise. The reaction mixture was heated at 40° C. overnight. An aqueous saturated NaHCO₃ solution (150 ml) was added to the reaction mixture. The reaction mixture was extracted three times with dichloromethane (150 ml) and the combined organic layers were dried, filtered and the solvent was removed by evaporation. The residue was isolated and purified using preparative thin layer chromatography with a mixture of dichloromethane/methanol (95:5) as eluent, yielding 210 mg of compound (A) (mp. 77° C.).

This compound (A) is known from U.S. Pat. No. 4,014,892 as compound (163).

### Example B.3

### Preparation of

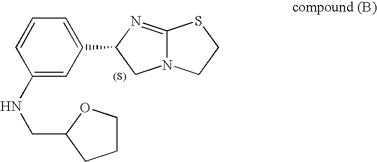

A solution of intermediate (7) (550 mg) was stirred in dry acetonitrile (30 ml) and NaHCO₃ (181 mg) was added. A solution of intermediate (9) in dry acetonitrile (15 ml) was added and after 15 minutes an amount of NaBH(OAc)₃ (547 mg) was added as a solid. After 2 hours TLC revealed that the reaction was complete. Saturated aqueous NaHCO₃ was added and the product was extracted twice with ethyl acetate. The combined ethyl acetate layers were dried and evaporated. The resulting residue was purified with flash column chromatography on silica using a mixture of dichloromethane and methanol in the following ratio and elution times: 10 minutes 3% methanol; 20 minutes 3% methanol to 5% methanol; 20 minutes 5% methanol with a flow rate of 30 ml/minute, yielding 237 mg of compound (B).

This compound (B) is known from U.S. Pat. No. 4,014,892 as compound (163) and from GB-1,365,515 as compound (81).

### C. Pharmacological Examples

### C.1. Efficacy Study of Anthelmintics in In Vivo H. contortus/Jirds Model

The anthelmintic efficacy of the compounds of the present invention was evaluated in an in vivo model using unmedicated jirds (Meriones unguiculatus), inoculated three times with approximately 300 exsheated infective larvae of Haemonchus contortus (multiresistant strain), treated orally with a test compound 11 days after their first infection with H. contortus larvae, and necropsied on day 14 to count the number of recovered H. contortus worms. The anthelmintic efficacy of the art known compounds (A) and (B) was also evaluated using the same model.

### Animals

Female CRW jirds aged between 28 and 35 days and weighing 30-35 g (Charles River, Sulzfeld, Germany) were used. Three jirds each were assigned randomly upon arrival to translucent polysulfone individually ventilated cages (48×37.5×21 cm) containing wood shavings. Commercial rodent chow and water were given ad libitum. Following a four-day acclimation period, the jirds were artificially infected.

### Parasite

A PolyRes strain of Haemonchus contortus (resistant against levamisole, mebendazole, ivermectin and closantel) was used. This strain has been maintained in artificially infected male donor lambs. Individual feces containing Haemonchus eggs were collected in fecal bags. The fecal pellets were broken, mixed with charcoal, moistened and put in an incubator for embryonation at 28° C. and 95% relative humidity. Seven days later this mixture was placed in Baermann funnels and third stage ensheathed larvae were collected after 12 hours. These larvae were rinsed with water for cleaning and disinfected with a 2% formalin solution. Such larvae can be used immediately for artificial infection or can be stored in the fridge at about 8° C. for a maximum duration of 6 months. Infective larvae (<6 months old) were exsheathed by rinsing with a 3.3 vol % commercial sodium hypochlorite solution during 10 minutes, filtered through a Buchner funnel, rinsed with water, concentrated in a Baermann funnel, and collected after 2 hours. Exsheathed larvae prepared in this manner can be used for subsequent infection of jirds or laid in a supply for extended periods by cooling in the gas over liquid nitrogen during 1 hour and storing in liquid nitrogen at −196° C.

### Infections

All jirds were inoculated orally with approximately 300 exsheathed infective larvae of H. contortus per dose on three consecutive days. Inoculations were administered using a blunted 18 G dosing needle fitted to a 1 ml syringe.

### Treatments

Eleven days after their first infection jirds were treated with test compounds, suspended or dissolved in 0.4 ml DMSO, and the dose to be tested was administered in a volume of 0.1 ml/50 g bodyweight via a blunted 18 G dosing needle fitted to a 1 ml syringe. Control animals, included in each experiment, remained untreated. Levamisole hydrochloride, mebendazole, ivermectin and closantel were used in different dosage titration experiments to validate the model.

### Necropsy

All jirds were starved 20 hours before necropsy and killed on day 14 past first infection by CO₂ inhalation. For worm recovery their stomachs were removed, opened longitudinally, and incubated in a beaker with 20 ml digestion fluid (10 g pepsin+30 ml concentrated hydrochloric acid) at 37° C. for 3 hours. Following digestion, the stomach content was passed through a tea-strainer, the passage fluid was catched in a sieve (32 μm) and the worms were recovered with tap water. The beakers were stored in the fridge for subsequent counting.

### Examination and Percentage Efficacy

The content of each beaker was mixed, poured over in a 6-well plate in 6 aliquots, and the worms were counted under an inverted microscope. The percentage efficacy for each test compound was determined and the results are summarized in Table 1 below.

TABLE 1
efficacy data in clearing PolyRes strain Haemonchus contortus from
jirds after oral treatment with a test compound

| | Dose | No. of | Med. | |
|---|---|---|---|---|
| Test Compound | mpk | Animals | W.b. | % Efficacy |
| Untreated Controls | 0 | 36 | 30.0 | — |
| Co. No. 1 | 2.5 | 3 | 36.0 | 65.9 |
| Co. No. 1 | 5 | 3 | 6.0 | 79.3 |
| Co. No. 1 | 10 | 6 | 0.0 | 100.0 |
| Co. No. 4 | 5 | 3 | 5.0 | 68.8 |
| Co. No. 4 | 10 | 3 | 3.0 | 85.0 |
| Co. No. A | 5 | 3 | 11.0 | 35.3 |
| Co. No. A | 10 | 3 | 7.0 | 65.0 |
| Co. No. B | 5 | 6 | 10.0 | 55.6 |
| Co. No. B | 10 | 3 | 14.0 | 60.6 |

Dose mpk: dose of the test compound in mg per kg body weight
No. of Animals: number of test animals
Med. W.b.: median worm burden
Percentage efficacy = ({(mean number of worms recovered from control group) − (mean number of worms recovered from treated group)} divided by (mean number of worms recovered from control group)) × 100

As can be seen in Table 1 both compounds (1) and (4) have a better demonstrated anthelmintic efficacy than the art known compounds (A) and (B).

1. A compound of formula (I) 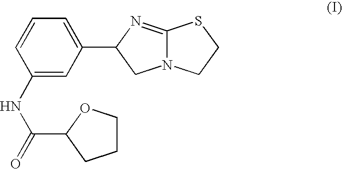
the pharmaceutically acceptable acid addition salts and the stereochemically isomeric forms thereof.

2. A compound as claimed in claim 1 wherein the 6-position of the 2,3,5,6-tetrahydro-imidazo[2,1-b]thiazole moiety has the (S)-configuration.

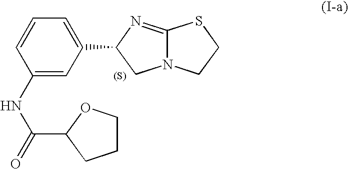

3. A compound as claimed in claim 2 wherein the compound is (2R)-tetrahydrofuran-2-carboxylic acid-(6S)-[3-(2,3,5,6-tetrahydro-imidazo[2,1-b]thiazol-6-yl)-phenyl]-amide and the pharmaceutically acceptable acid addition salts thereof.

4. A compound as claimed in claim 2 wherein the compound is (2S)-tetrahydrofuran-2-carboxylic acid-(6S)-[3-(2,3,5,6-tetrahydro-imidazo[2,1-b]thiazol-6-yl)-phenyl]-amide and the pharmaceutically acceptable acid addition salts thereof.

5. A pharmaceutical composition comprising a pharmaceutically acceptable carrier and a therapeutically active amount of a compound according to claim 1.

6. (canceled)

7. (canceled)

8. (canceled)

9. A composition comprising as a first active ingredient a compound as claimed in any of claim 1 and as a second active ingredient another anthelmintic or antiparasitic agent.

10. A process for preparing a compound of formula (I) wherein an intermediate of formula (V) is reacted with an intermediate of formula (IV) in a suitable reaction-inert solvent 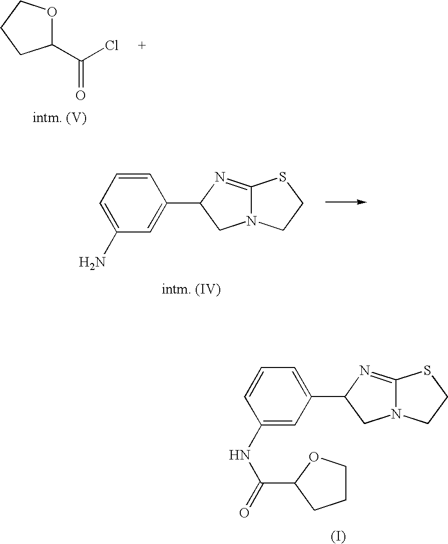
or if desired; a compound of formula (I) is converted into a pharmaceutically acceptable acid addition salt, or conversely, an acid addition salt of a compound of formula (I) is converted into a free base form with alkali; and, if desired, preparing stereochemically isomeric forms thereof.

11. A method for the treatment, control and prevention of endo- and ectoparasite infections, comprising administering to a mammal in need thereof an effective amount of a compound of claim 1.
